(19) United States
(12) Patent Application Publication
(10) Pub. No.: US 2018/0221252 A1
(43) Pub. Date: Aug. 9, 2018

(54) Sun protection towelettes

(71) Applicant: Franklin Wang, La Canada, CA (US)

(72) Inventor: Franklin Wang, La Canada, CA (US)

(21) Appl. No.: 15/429,066

(22) Filed: Feb. 9, 2017

(51) Int. Cl. A61K 8/02; A61Q 17/04

(52) U.S. Cl. CPC A61K 8/0208; A61K 2800/87; A61K 2800/56; A61Q 17/04

(57) ABSTRACT

This invention relates to a package containing one or more sun protection towelettes moistened or pre-saturated with sunscreen with different values of SPF for application on skin of users. The one or more sun protection towelettes are pre-saturated with stabled UV sunscreen having long shelf life. The one or more sun protection towelettes are made up of soft and non-abrasive paper or fabric.

### CROSS REFERENCE TO RELATED APPLICATIONS

Not applicable

### FEDERALLY SPONSORED RESEARCH AND DEVELOPMENT

Not applicable.

### MICROFICHE

Not applicable

### BACKGROUND OF THE INVENTION

### (1) Field of the Invention

The present invention relates to the field of sun protection towelettes. The invention, particularly relates to sun protection towelettes impregnated with sunscreen agents and contained in a single convenient package.

### (2) Background of the Invention

A variety of pre-moistened wipes are utilized for the purposes of cleaning both households; and the human body. Furthermore, some of these wipes are also used to moisturize the face. There are water-moistened, soluble pre-moistened wipes available on the commercial market, however, the purpose of these products is specifically for cleansing for infants, or wiping dirt off face and general bodily hygiene. Currently, there are pre-moistened, sunscreen wipes and towelettes available commercially in the form of small hand held sizes. There are also pre-sunscreen moistened sunscreen applicators or towelettes that are biodegradable available in market. Furthermore, existing sunscreen wipe products are not available in bulk in case sunscreen has to be applied hard to reach places, such as one's back, without assistance. The closest method of self-application for difficult to reach areas is provided by spray mechanisms that disperse sunscreen which often partially miss the area for which the sunscreen is intended for.

Also, one of the greatest problems with existing sunscreen wipes is the issue of disposability and ecological value. The existing sunscreen wipe's “fabrics” are made of synthetic materials that do not break down. They must be thrown away in a garbage receptacle in order to be disposed of.

A number of different types of sunscreen towelettes are available in prior art. For example, see: U.S. patent application Ser. No. 09/841,208 to Gott et al.; Ser. No. 10/304,681 to Malcolm et al.; Ser. No. 11/498,385 to Persefeld et al.; Ser. No. 13/874,481 to Whitmire et al.; U.S. Pat. No. 6,054,120 to Burguyne et al.

Prior art U.S. patent application Ser. No. 09/841,208 discloses a cosmetic towelette which includes a water-insoluble substrate and a fluid cosmetic composition impregnated into the substrate. The composition has a viscosity ranging from about 1 cps to 10,000 cps. The composition includes a water phase, a sunscreen phase and a surfactant system. The sunscreen phase is immiscible with the water phase and contains at least 25% organic sunscreen agent. Towelettes impregnated with the composition impart an effective SPF to the skin when applied thereto.

Another Prior art U.S. patent application Ser. No. 11/498,385 discloses a packeted towelette impregnated with body lotion is disclosed. The towelette is packaged in a leak proof packet and fits easily into a pocket, purse, suitcase, briefcase, golf bag, gym bag or other container. The body lotion contained within the packet is a mixture of many ingredients which, when placed on the skin, helps moisturize and protect the skin from the ravages of the environment.

Therefore, there is a need for disposable, biodegradable sunscreen towelettes that provide easy to use, one step applications to all areas of the body, are small in size and available in bulk.

### SUMMARY OF THE INVENTION

In the view of the foregoing disadvantages inherent in the known types of sun protection towelettes now present in the prior art, the present invention provides an improved sun protection towelettes. As such, the general purpose of the present invention, which will be described subsequently in greater detail, is to provide a new and improved sunscreen moistened sun protection towelettes which has all the advantages of the prior art and none of the disadvantages.

An object of the invention is to provide one or more sun protection towelettes moistened or pre-saturated with sunscreen with different values of SPF for application on skin of users.

It is another object of the present invention to provide one or more sun protection towelettes moistened with sunscreen packaged in a package.

It is another object of the present invention to provide one or more sun protection towelettes having base material made up of soft and non-abrasive paper or fabric.

It is another object of the present invention to provide one or more sun protection towelettes that are not easy to rip apart.

It is another object of the present invention to provide one or more sun protection towelettes pre-saturated with stabled UV sunscreen having long shelf life.

It is another object of the present invention to provide a sunscreen that performs functions such as moistening, whitening, tanning and anti-aging.

It is another object of the present invention to provide one or more sun protection towelettes that are disposable.

It is another object of the present invention to provide one or more sun protection towelettes that are bacteria free.

In this respect, before explaining at least one embodiment of the invention in detail, it is to be understood that the invention is not limited in its application to the details of construction and to the arrangements of the components set forth in the following description or illustrated in the drawings. The invention is capable of other embodiments and of being practiced and carried out in various ways. Also, it is to be understood that the phraseology and terminology employed herein are for the purpose of description and should not be regarded as limiting.

These together with other objects of the invention, along with the various features of novelty which characterize the invention, are pointed out with particularity in the disclosure. For a better understanding of the invention, its operating advantages and the specific objects attained by its uses, reference should be had to the accompanying drawings and descriptive matter in which there are illustrated preferred embodiments of the invention.

### BRIEF DESCRIPTION OF THE DRAWINGS

The invention will be better understood and objects other than those set forth above will become apparent when consideration is given to the following detailed description thereof. Such description makes reference to the annexed drawings wherein:

FIG. 1 depicts front view of a package containing one or more sun protection towelettes; and

FIG. 2 depicts front view of one or more sun protection towelettes.

### DETAILED DESCRIPTION OF THE INVENTION

In the following detailed description, reference is made to the accompanying drawings which form a part hereof and in which is shown by way of illustration specific embodiments in which the invention may be practiced. These embodiments are described in sufficient detail to enable those skilled in the art to practice the invention, and it is to be understood that the embodiments may be combined, or that other embodiments may be utilized and that structural, electrical and logical changes may be made without departing from the spirit and scope of the present invention. The following detailed description is, therefore, not to be taken in a limiting sense, and the scope of the present invention is defined by the appended claims and their equivalents.

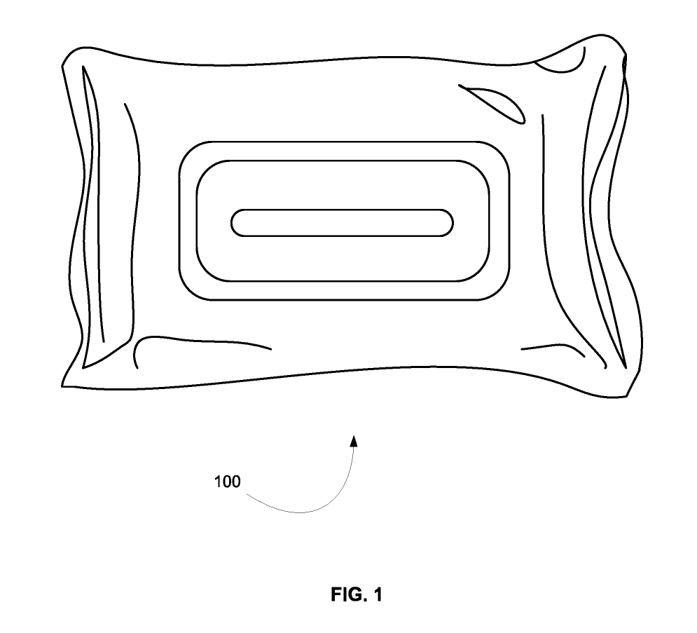

FIG. 1 depicts front view of a package 100 containing one or more sun protection towelettes 102 (not shown in FIG. 1) according to an embodiment herein. The package 100 is provided to apply UV protective lotion to skin that is different from the sunscreen present in the art. The package 100 contains plain one or more sun protection towelettes 102 with different softness. The one or more sun protection towelettes 102 are pre-saturated with different screen lotions or products by various manufacturers and supplier.

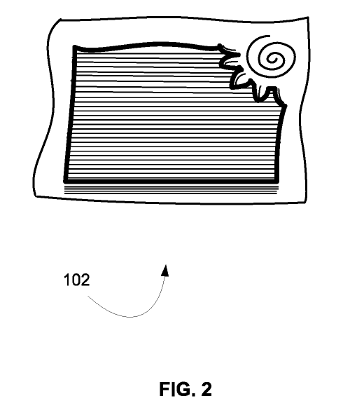

FIG. 2 depicts front view of one or more sun protection towelettes 102 according to an embodiment herein. The one or more sun protection towelettes 102 are pre moistened using a screening lotion or gel. The screening gel or lotion is a sunscreen lotion. The one or more sun protection towelettes 102 are moistened with sunscreen lotions of different SPF values. The one or more sun protection towelettes 102 are basically an applicator. The lotion is impregnated onto the one or more sun protection towelettes 102 when applied becomes a layer of sun protection to a desired SPF. The one or more sun protection towelettes 102 are evenly coated with sunscreen. The one or more sun protection towelettes 102 is comprised of a base material made up of soft and non-abrasive paper or fabric, such that the towelettes are not easy to rip apart.

The lotion used is stabled UV sunscreen that has long shelf life. The sunscreen performs different functions including moistening, whitening tanning anti-aging or the like. Using the one or more sun protection towelettes 102 of present invention users can apply sunscreen without using touching the skin by fingers or palm. The one or more sun protection towelettes 102 have small size for example the size of visiting cards. One or more sun protection towelettes 102 having impregnated or pre-saturated or moistened with sunscreen are readily packaged in a package 100. The package 100 containing the one or more sun protection towelettes 102 is easy to carry. The one or more sun protection towelettes 102 of the present invention are easy to use and apply sunscreen, disposable, bacteria free, airport checkpoint safe, inexpensive and can be used for applying sunscreen on body of infants or babies.

It is to be understood that the above description is intended to be illustrative, and not restrictive. For example, the above-discussed embodiments may be used in combination with each other. Many other embodiments will be apparent to those of skill in the art upon reviewing the above description.

The benefits and advantages which may be provided by the present invention have been described above with regard to specific embodiments. These benefits and advantages, and any elements or limitations that may cause them to occur or to become more pronounced are not to be construed as critical, required, or essential features of any or all of the embodiments.

While the present invention has been described with reference to particular embodiments, it should be understood that the embodiments are illustrative and that the scope of the invention is not limited to these embodiments. Many variations, modifications, additions and improvements to the embodiments described above are possible. It is contemplated that these variations, modifications, additions and improvements fall within the scope of the invention.

What is claimed is:

1) A package for containing cleaning material, wherein said package comprises:
a plurality of sun protection towelettes; wherein said plurality of sun protection towelettes are pre-moistened.

2) The package of claim 1, wherein said plurality of sun protection towelettes are pre-moistened with a screening lotion.

3) The package of claim 2, wherein said screening lotion is a sunscreen lotion.

4) The package of claim 3, wherein said sunscreen lotion is impregnated with different values of Sun Protection Factor.

5) The package of claim 3, wherein said sunscreen lotion is a stable UV sunscreen.

6) The package of claim 1, wherein said plurality of sun protection towelettes are made up of soft and non-abrasive material.

7) The package of claim 6, wherein said non-abrasive material is selected from a group of paper or fabric.

8) The package of claim 1, wherein said plurality of sun protection towelettes are disposable.

9) The package of claim 1, wherein said plurality of sun protection towelettes are biodegradable.

10) The package of claim 1, wherein said plurality of sun protection towelettes are bacteria free.

11) A package for containing cleaning material, wherein said package comprises:
a plurality of disposable, soft sun protection towelettes; wherein said plurality of sun protection towelettes are pre-moistened with a stable sun screening lotion, wherein said plurality of sun protection towelettes are biodegradable and bacteria free.
